(19) United States
(12) Patent Application Publication
(10) Pub. No.: US 2006/0051401 A1
(43) Pub. Date: Mar. 9, 2006

(54) Controlled nanofiber seeding

(75) Inventors: Sanjeev K. Manohar, Dallas, TX (US); Xinyu Zhang, Richardson, TX (US)

Correspondence Address: CHALKER FLORES, LLP, 2711 LBJ FRWY, Suite 1036, DALLAS, TX, 75234, US

(73) Assignee: Board Of Regents, The University Of Texas System, Austin, TX (US)

(21) Appl. No.: 10/936,437

(22) Filed: Sep. 7, 2004

(51) Int. Cl. A61K 9/70

(52) U.S. Cl. 424/443

(57) ABSTRACT

The present invention includes compositions and methods for the controlled formation of nano and micropolymers in a single-step that includes a structural substrate, an oxidatively reactive monomer and an oxidant to form a polymer that takes the morphology of the structural substrate.

### TECHNICAL FIELD OF THE INVENTION

The present invention relates in general to the field of controlled nanofiber formation, and more particularly, to compositions and methods for the synthesis of polymers with a pre-selected morphology.

### BACKGROUND OF THE INVENTION

Without limiting the scope of the invention, its background is described in connection with nanopolymer formation.

One such nanopolymer is taught in U.S. Pat. No. 5,334,292, issued to Rajeshwar, et al., entitled, “Conducting polymer films containing nanodispersed catalyst particles: a new type of composite material for technological applications.” The invention is directed to an electronically conductive polymer film comprising colloidal catalytic particles homogeneously dispersed therein. The electronically conductive polymer is preferably polypyrrole although other conductive polymers, for example, polyaniline and polythiophene are also used. The preferred catalytic particles are platinum although other catalytic particles such as RuO₂, Ag, Pd, Ni, Cd, Co, Mo, Mn-oxide, Mn-sulfide, a molybdate, a tungstate, tungsten carbide, a thiospinel, Ru, Rh, Os, Ir, or a platinum palladium alloy (Pt/Pd). The colloidal catalytic particles incorporated in the film of the present invention are less than 100 nanometers in size, preferably about 10 nm in size. In one preferred composition the polymer is polypyrrole and the catalytic particles are platinum. The inventors also teach a method of producing an electronically conductive polymer film containing colloidal catalytic particles homogeneously dispersed therein. This method includes preparing a colloidal suspension of catalytic particles in a solution comprising an electronically conductive polymer precursor.

Yet another example of a nanopolymer is taught in U.S. Pat. No. 6,712,917, issued to Gash, et al., entitled, “Inorganic metal oxide/organic polymer nanocomposites and method thereof.” Briefly, a synthetic method for preparation of hybrid inorganic/organic energetic nanocomposites is disclosed. The method employs the use of stable metal inorganic salts and organic solvents as well as an organic polymer with good solubility in the solvent system to produce novel nanocomposite energetic materials. In addition, fuel metal powders (particularly those that are oxophillic) can be incorporated into composition. The materials taught is said to be characterized by thermal methods, energy-filtered transmission electron microscopy (EFTEM), N₂ adsoprtion/desorption methods, and Fourier-Transform (FT-IR) spectroscopy. According to these characterization methods the organic polymer phase fills the nanopores of the composite material, providing superb mixing of the component phases in the energetic nanocomposite.

Another example of prior art nanopolymer formation is taught in U.S. Pat. No. 6,746,825, issued to Nealey, et al., entitled “Guided self-assembly of block copolymer films on interferometrically nanopatterned substrates.” Briefly, the copolymer structures are formed by exposing a substrate with an imaging layer thereon to two or more beams of selected wavelengths to form interference patterns at the imaging layer to change the wettability of the imaging layer in accordance with the interference patterns. A layer of a selected block copolymer is deposited onto the exposed imaging layer and annealed to separate the components of the copolymer in accordance with the pattern of wettability and to replicate the pattern of the imaging layer in the copolymer layer. Stripes or isolated regions of the separated components may be formed with periodic dimensions in the range of 100 nm or less.

### SUMMARY OF THE INVENTION

The present invention includes compositions and methods of controlled polymer formation that includes a single-step polymerization on or about a structural substrate with an oxidatively reactive monomer and an oxidant to form a polymer that takes the morphology of the structural substrate. For example, the structural substrate may be an active substrate. The oxidatively reactive monomers may be polymerized onto a bio-compatible polymer, a bio-degradable polymer or even a biodegradable and bio-compatible polymer. Examples of oxidatively reactive monomer for use with the present invention includes: lactic acid, glycolic-lactic acid, glycolic acid, cyanobutylacrylate, propylbutyl, pyrrole, 3,4-ethylenedioxythiophene, aniline, and combinations thereof. The nano and microparticles, fibers and other structures may also be formed from one or more of the following polymerizable monomers: ethylene glycol; ethylene oxide; partially or fully hydrolyzed vinyl alcohol; vinylpyrrolidone; ethyloxazoline; ethylene oxide-co-propylene oxide; poloxamers, meroxapols, poloxamines; conductive polymers; methyl, lauryl, stearyl or butyl methacrylates; vinyl halides; tetrafluroethylene, acrylonitrile, ethylene; methyl, ethyl or butyl acrylates and combinations thereof.

Additional polymers may be formed and/or added to the morphologically controlled nanopolymers formed herein. Examples of additional polymers include: natural polymers such as carboxymethyl cellulose, and hydroxyalkylated celluloses such as hydroxyethyl cellulose and methylhydroxypropyl cellulose, polypeptides, polysaccharides or carbohydrates, polysucrose, hyaluronic acid, dextran, heparan sulfate, chondroitin sulfate, heparin, and alginate, and proteins such as gelatin, collagen, albumin, and ovalbumin, other copolymers, and combinations thereof. One specific embodiment of the present invention includes the addition of a bioactive compound. Examples of bioactive compound includes one or more drugs, proteins, peptides, polysaccharides, oligonucleotides (RNA, DNA, PNA or combinations thereof), aptamers, antibiotics, anti-cancer drugs, antigens, antibodies, bioactive extracts, synthetic organic molecules and/or synthetic inorganic molecules.

Structural substrates for use with the present invention include, e.g., an inorganic molecule, a crystal, a magnet, a metal, an isolator, a conductor, a semiconductor, a nanotube, a nanosphere, a nanosheet, a nanofilm, a C₆₀ fullerenes, a fullerene-type concentric graphitic particle, a semiconductor, CdSe, CdS, ZnS, GaAs, InP, nanowires/nanorods such as Si, Ge, SiO_{X}, Ge, O_{X}, nanotubes, single or multiple elements such as carbon, B_{X}N_{y}, C_{X}, B_{Y}, N_{Z}, MoS₂, and WS₂ and combinations thereof. The structural substrate may even be an organic molecule, a peptide, a surfactant, a lipid, a protein, a carbohydrate, a nucleic acid, a metal, a plastic, a monomer, a dimer, a trimer, an oligomer, a polymer, a pharmaceutical, a nutraceutical, a cosmoceutical and combinations thereof. Specific examples of structural substrates for use as seeding molecules for the polymerization reaction include, e.g., emeraldine•HCl nanofiber, a HiPco single-walled carbon nanotubes (SWNT), a hexapeptide AcPHF6, a V₂O₅ nanofiber, a polyaniline dimer, polypyrrole dimer and combinations thereof. The present invention includes a product made with the above method or process.

In another embodiment, the present invention is a molecular polymer made from oxidatively coupled monomers, wherein the polymer is formed on or about a molecular seed and the polymer takes on the morphology of the molecular seed. The polymer may be electrically conducting, bio-compatible, bio-degradable or even bio-degradable and bio-compatible. The polymer may be formed into, e.g., a film, a sheet, a pill, a powder, a matrix, a fiber, a pad, a filter, a sheet, a sensor, an insert, a cathode, an anode, a passivation, a semiconductor and combinations thereof. The molecular polymer may be made from, e.g., lactic acid, glycolic-lactic acid, glycolic acid, cyanobutylacrylate, propylbutyl, pyrrole, ethylenedioxythiophene and combinations thereof. The polymer may be formed from and/or to include a bioactive compound, e.g., a drug, a protein, a peptide, a polysaccharide, an oligonucleotide, an antibiotic, an anti-cancer drug, an antigen, an antibody, a bioactive extract, a synthetic organic molecule, and a synthetic inorganic molecule. The polymer may be formed on or about an inorganic molecule, a crystal, a magnet, a metal, an insulator, a conductor, a single crystal semiconductor, a semiconductor, a nanotube, a nanosphere, a nanosheet, a nanofilm, a nanocone, a C₆₀ fullerenes, a fullerene-type concentric graphitic particle, sheet, cone, tube, rod; a semiconductor, CdSe, CdS, ZnS, GaAs, InP, nanowires/nanorods such as Si, Ge, SiO_{X}, Ge, O_{X}, nanotubes, single or multiple elements such as carbon, B_{X}N_{y}, C_{X}, B_{Y}, N_{Z}, MoS₂, and WS₂ and combinations thereof. In one example, the polymer is formed on or about an organic molecule, a peptide, a surfactant, a lipid, a protein, a carbohydrate, a nucleic acid, a metal, a plastic, a monomer, a dimer, a trimer, an oligomer, a polymer, a pharmaceutical, a nutraceutical, a cosmoceutical and combinations thereof. The molecular seed itself may be an oxidant, an inorganic molecule, a crystal, a magnet, a metal, an insulator, a conductor, a semiconductor, a nanotube, a nanosphere, a nanosheet, a nanofilm, a C₆₀ fullerenes, a fullerene-type concentric graphitic particle, a semiconductor, CdSe, CdS, ZnS, GaAs, InP, nanowires/nanorods such as Si, Ge, SiO_{X}, Ge, O_{X}, nanotubes, single or multiple elements such as carbon, BₓN_{y}, C_{X}, B_{Y}, N_{Z}, MoS₂, and WS₂ and combinations thereof.

In another embodiment, the polymerization method includes the step of polymerizing in a single step an oxidatively reactive monomer in the presence of an oxidant on or about a molecular seed, wherein the polymer takes on the morphology of the structural substrate.

Alternatively, the method of controlled nanofibril formation may include polymerizing an oxidatively reactive monomer on or about a template in the presence of an oxidant, wherein the polymer formed takes on the morphology of the structural substrate. When formed into a nanofiber, the seeded polymer may have a surface area greater than 51 m²/g. When formed, the nanoparticle, sheet or film may be, e.g., electrically conducting or even insulating. For example, alternating sheets of conductive and non-conductive films may be formed to create electronic capacitors, batteries, switches, transistors and the like. One advantage of the present invention is that it allows for the synthesis of nano and micro structures of controlled morphology into, e.g., a film, a sheet, a pill, a powder, a matrix, a fiber, a pad, a filter, a sheet, a sensor, an insert, a cathode, an anode, a passivation, a semiconductor and combinations thereof. When used in a biological, environmental or like setting, the nanostructures formed with the methods disclosed herein may be targeted to one or more specific locations using, e.g., a targeting moiety.

### BRIEF DESCRIPTION OF THE DRAWINGS

For a more complete understanding of the features and advantages of the present invention, reference is now made to the detailed description of the invention along with the accompanying figures and in which:

FIG. 1A through 1D are SEM images of emeraldine•HCl nanofibers synthesized by seeding the reaction using the following: (Figure A) 1.5 mg of emeraldine•HCl nanofibers (SEM image inset), (FIG. 1B) 1.6 mg of HiPco SWNT (SEM image inset), (FIG. 1C) 1.0 mg of the hexapeptide AcPHF6 (TEM image inset), and (FIG. 1D) 4 mg of V₂O₅ nanofibers (SEM image inset);

FIGS. 2A to 2D are high magnification images of emeraldine•HCl nanofibers by seeding the reaction with: (FIG. 2A) 1.5 mg emeraldine•HCl nanofibers (SEM images); (FIG. 2B) 1.6 mg HiPco SWNT (SEM images); (FIG. 2C) 1.0 mg hexapeptide AcPHF6 (TEM images); and (FIG. 2D) 4 mg V₂O₅ nanofibers (SEM images);

FIG. 3 is an SEM image of emeraldine•HCl nanofibers using an unseeded (conventional) chemical polymerization;

FIG. 4 is an SEM image of emeraldine•HCl powder on glass slides synthesized using a unseeded (conventional) chemical polymerization;

FIG. 5 is an SEM image of nanoparticles of emeraldine•HCl nanospheres synthesized using a seeded reaction of doped polypyrrole•Cl using FeCl₃ as an oxidant;

FIG. 6 is a TEM image of emeraldine•HCl nanofibers using a seeded reaction using SWNT;

FIG. 7A is an SEM image (left) of an in-situ deposited film of emeraldine•HCl nanofibers on a glass microscope slide synthesized using 1.6 mg of SWNT seed template;

FIG. 7B is a graph of the solid-state UV/vis spectra and optical images of films of emeraldine•HCl (green, curve 1) and emeraldine base (blue, curve 2) on a glass microscope slide;

FIG. 8 is a graph that shows the charge/discharge capacity plot of emeraldine•HCl powder in the range 0.4-0.5 V (vs SCE) in aqueous 1.0 M camphorsulfonic acid electrolyte; Charge (curve A), discharge (curve B) cycles for nanofibers and charge (curve C), discharge (curve D) cycles for conventional (nonfibrillar) polyaniline (Inset: cyclic voltammograms of polyaniline nanofibers (outer plot) and conventional polyaniline (inner plot)).

FIGS. 9A and 9B are SEM images of nanofibers of (9A) emeraldine•HCSA and (9B) emeraldine•AMPSA synthesized in the presence of TX100 (inset: conventional synthesis, without TX100);

FIGS. 10A and 10B are TEM images of the nanofibers of the present invention obtained after moderate mechanical agitation;

FIG. 11 is a graph that shows a surface tension versus concentration plot for TX100 in aqueous 1.0M HCl (▪), AMPSA (●), AMPSA plus aniline (▴), HCSA (▾), and HCSA plus aniline (♦);

FIGS. 12A through 12D are SEM images of polypyrrole.Cl (insets: seed template): (12A) unseeded reaction; (12B) seeded with 1.5 mg HiPco SWNT; (12C) seeded with 4 mg of V₂O₅; and (12D) seeded with SWNT pre-exposed to (NH₄)₂S₂O₈;

FIG. 13A through 13D are SEM images of polypyrrole.Cl: (13A) bulk powder, seeded by pernigraniline; (13B) bulk powder, seeded by aniline dimer; (13C) film on glass, seeded by V₂O₅; and (13D) film on poly(ethyleneterephthalate), PET seeded by V₂O₅; and

FIGS. 14A and 14B are SEM images of polypyrrole•Cl nanofibers synthesized in ethanol/FeCl₃ using V₂O₅ as the seed, prior to the reaction V₂O₅ was stirred in ethanol for (14A) 30 minutes and (14B) 12 hours.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “nanostructure” is used to describe materials such as nanoparticles, nanofibrils, nanoshells, nanosheets, nanotubes, nanofilms, nanorods and the like, e.g., C₆₀₋₁₂₀ fullerenes, fullerene-type concentric graphitic particles, sheets, rods, cones; metal; compound semiconductors such as CdSe, CdS, ZnS, GaAs, InP; nanowires/nanorods such as Si, Ge, SiO_{X}, Ge, Ox; or nanotubes composed of either single or multiple elements such as carbon, B_{X}N_{y}, C_{X}, B_{Y}, N_{Z}, MoS₂, and WS₂. One of the common features of nanostructure materials is their basic building blocks, e.g., a nanoparticle or a carbon nanotube may have has a dimension that is less than about 500 nm in at least one direction. As used herein, the term “nanostructural material” is used to describe materials that are composed entirely, or almost entirely of nanostructure materials, as well as materials composed of both nanostructures as well as other types of materials, thereby forming a composite construction. As used herein, the term “nanofiber” is used to describe a fiber, whether covalently or ionically attached that is less between about 500 to 1000 nm in at least one direction.

As used herein, the term “microstructure” is used to describe materials such as microparticles, microfibrils, microshells, microsheets, microtubes and the like, in which the following nanostructures are used as the structural templates for the formation of the controlled polymers that take on the morphology of the template, e.g., C₆₀₋₁₂₀ fullerenes, fullerene-type concentric graphitic particles; metal; compound semiconductors such as CdSe, InP; nanowires/nanorods such as Si, Ge, SiO_{X}, Ge, Ox; or nanotubes composed of either single or multiple elements such as carbon, B_{X}N_{y}, C_{X}, B_{Y}, N_{Z}, MoS₂, and WS₂. One of the common features of nanostructure materials is their basic building blocks, e.g., a nanoparticle or a carbon nanotube may have has a dimension that is less than about 500 nm in at least one direction. As used herein, the term “microstructural material” is used to describe materials that are composed entirely, or almost entirely of nanostructure materials, as well as materials composed of both nanostructures as well as other types of materials, thereby forming a composite construction. As used herein, the term “microfiber” is used to describe a fiber, whether covalently or ionically attached that is less between about 1 to 1000 micrometers in at least one direction.

As used herein, the term “template,” and “structural template” is used to describe the physical shape around which the nano and micropolymers of the present invention are formed. As used herein, the term “active template” is used to describe the physical shape around which the nano and micropolymers of the present invention are formed but also serves as the activating agent for initial polymer formation, e.g., V₂O₅. Another term used herein is “sacrificial template” which is defined as a template that may be consumed, exhausted or removed following the formation of the nano or micropolymers of the present invention. The sacrificial template may be, e.g., V₂O₅, in which case its shape, size and/or amount is selected so as to be exhausted during the initiation and formation of the polymer. Alternatively, the sacrificial template may be removed using chemical, electrical or other methods and combinations thereof to leave gaps and/or holes within the nano or micropolymer.

As used herein, the term “seeding” is used to describe the use of small molecules, di-mers, tri-mers, oligomers, polymers, crystals, fibers, strings, films and the like that are used to initiate the formation of the morphologically controlled polymers of the present invention. As used herein, the term “molecular seed” is used to describe small molecules, di-mers, tri-mers, oligomers and the like that are used to initiate the formation of the morphologically controlled polymers of the present invention. Examples of molecular seeds include: an inorganic molecule, a crystal, a magnet, a metal, an insulator, a conductor, a semiconductor, a nanotube, a nanosphere, a nanosheet, a nanofilm, a C₆₀ fullerenes, a fullerene-type concentric graphitic particle, a semiconductor, CdSe, CdS, ZnS, GaAs, InP, nanowires/nanorods such as Si, Ge, SiO_{X}, Ge, O_{X}, nanotubes, single or multiple elements such as carbon, B_{X}N_{y}, C_{X}, B_{Y}, N_{Z}, MoS₂, and WS₂ and combinations thereof.

As used herein, the term “oxidant” is used to describe molecules that are used to activate and catalyze the polymerization reactions of the present invention. Examples of oxidants include: ammonium peroxydisulfate, FeCl₃, H₂O₂, organic peroxides, and even V₂O₅.

As used herein, “nanoparticle” is defined as a particle having a diameter of from 1 to 1000 nanometers, having any size, shape or morphology. The nanoparticle may even be a “nanoshell,” which is a nanoparticle having a discrete dielectric or semiconducting core section surrounded by one or more conducting shell layers. A “nanoshell” is a subspecies of nanoparticles characterized by the discrete core/shell structure. Both nanoshells and nanoparticles may contain dopants for binding to, e.g., negatively charged molecules such as DNA, RNA and the like. Examples of commonly used, positively charged dopands include Pr⁺³, Er⁺³, and Nd⁺³. As used herein, “shell” means one or more shells that will generally surround at least a portion of one core. Several cores may be incorporated into a larger nanoshell.

As used herein, the term “targeting moiety,” is used to describe molecules capable of specifically binding to a particular target molecule and forming a bound complex as described above. Thus, the ligand and its corresponding target molecule form a specific binding pair.

As used herein, “pharmaceutically” and/or “pharmacologically acceptable” refer to molecular entities and/or compositions that do not produce an adverse, allergic and/or other untoward reaction when administered to an animal, as appropriate.

As used herein, “pharmaceutically acceptable carrier” may include any and/or all solvents, dispersion media, coatings, antibacterial and/or antiflngal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For administration, preparations should meet sterility, pyrogenicity, general safety and/or purity standards as required by FDA Office of Biologics standards.

As used herein, the term “therapeutically effective dosage” is used to describe the amount that reduces the amount of symptoms of the condition in the infected subject by at least about 20%, more preferably by at least about 40%, even more preferably by at least about 60%, and still more preferably by at least about 80% relative to untreated subjects. Often, for pediatric doses the amount will be half or less of the adult dose. For example, the efficacy of a compound may be evaluated in an animal model system that may be predictive of efficacy in treating the disease in humans. Bioactive compounds are administered at a therapeutically effective dosage sufficient to treat a condition associated with a condition in a subject.

As used herein, the term “active ingredient(s),” “pharmaceutical ingredient(s),” “active agents” and “bioactive agent” are defined as drugs and/or pharmaceutically active ingredients. The present invention may be used to encapsulate, attach, bind or otherwise be used to affect the storage, stability, longevity and/or release of any of the following drugs as the pharmaceutically active agent in a composition.

Bioactive Agents

One or more of the following bioactive agents may be combined with one or more carriers and the present invention (which may itself be the carrier):

Analgesic anti-inflammatory agents such as, acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like.

Drugs having an action on the central nervous system, for example sedatives, hypnotics, antianxiety agents, analgesics and anesthetics, such as, chloral, buprenorphine, naloxone, haloperidol, fluphenazine, pentobarbital, phenobarbital, secobarbital, amobarbital, cydobarbital, codeine, lidocaine, tetracaine, dyclonine, dibucaine, cocaine, procaine, mepivacaine, bupivacaine, etidocaine, prilocalne, benzocaine, fentanyl, nicotine, and the like. Local anesthetics such as, benzocaine, procaine, dibucaine, lidocaine, and the like.

Antihistaminics or antiallergic agents such as, diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, clorprenaline, terfenadine, chlorpheniramine, and the like. Anti-allergenics such as, antazoline, methapyrilene, chlorpheniramine, pyrilamine, pheniramine, and the like. Decongestants such as, phenylephrine, ephedrine, naphazoline, tetrahydrozoline, and the like.

Antipyretics such as, aspirin, salicylamide, non-steroidal anti-inflammatory agents, and the like. Antimigrane agents such as, dihydroergotamine, pizotyline, and the like. Acetonide anti-inflammatory agents, such as hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodiun, tolmetin, and the like. Muscle relaxants such as, tolperisone, baclofen, dantrolene sodium, cyclobenzaprine.

Steroids such as, androgenic steriods, such as, testosterone, methyltestosterone, fluoxymesterone, estrogens such as, conjugated estrogens, esterified estrogens, estropipate, 17-β estradiol, 17-β estradiol valerate, equilin, mestranol, estrone, estriol, 17β ethinyl estradiol, diethylstilbestrol, progestational agents, such as, progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17-α hydroxyprogesterone, dydrogesterone, dimethisterone, ethinylestrenol, norgestrel, demegestone, promegestone, megestrol acetate, and the like.

Respiratory agents such as, theophilline and β₂-adrenergic agonists, such as, albuterol, terbutaline, metaproterenol, ritodrine, carbuterol, fenoterol, quinterenol, rimiterol, solmefamol, soterenol, tetroquinol, and the like. Sympathomimetics such as, dopamine, norepinephrine, phenylpropanolamine, phenylephrine, pseudoephedrine, amphetamine, propylhexedrine, arecoline, and the like.

Antimicrobial agents including antibacterial agents, antifungal agents, antimycotic agents and antiviral agents; tetracyclines such as, oxytetracycline, penicillins, such as, ampicillin, cephalosporins such as, cefalotin, aminoglycosides, such as, kanamycin, macrolides such as, erythromycin, chloramphenicol, iodides, nitrofrantoin, nystatin, amphotericin, fradiomycin, sulfonamides, purroInitrin, clotrimazole, miconazole chloramphenicol, sulfacetamide, sulfamethazine, sulfadiazine, sulfamerazine, sulfarnethizole and sulfisoxazole; antivirals, including idoxuridine; clarithromycin; and other anti-infectives including nitrofurazone, and the like.

Antihypertensive agents such as, clonidine, α-methyldopa, reserpine, syrosingopine, rescinnamine, cinnarizine, hydrazine, prazosin, and the like. Antihypertensive diuretics such as, chlorothiazide, hydrochlorothrazide, bendoflumethazide, trichlormethiazide, furosemide, tripamide, methylclothiazide, penfluzide, hydrothiazide, spironolactone, metolazone, and the like. Cardiotonics such as, digitalis, ubidecarenone, dopamine, and the like. Coronary vasodilators such as, organic nitrates such as, nitroglycerine, isosorbitol dinitrate, erythritol tetranitrate, and pentaerythritol tetranitrate, dipyridamole, dilazep, trapidil, trimetazidine, and the like. Vasoconstrictors such as, dihydroergotamine, dihydroergotoxine, and the like. β-blockers or antiarrhythmic agents such as, timolol pindolol, propranolol, and the like. Humoral agents such as, the prostaglandins, natural and synthetic, for example PGE₁, PGE₂α, and PGF₂α, and the PGE₁ analog misoprostol. Antispasmodics such as, atropine, methantheline, papaverine, cinnamedrine, methscopolamine, and the like.

Calcium antagonists and other circulatory organ agents, such as, aptopril, diltiazem, nifedipine, nicardipine, verapamil, bencyclane, ifenprodil tartarate, molsidomine, clonidine, prazosin, and the like. Anti-convulsants such as, nitrazepam, meprobamate, phenyloin, and the like. Agents for dizziness such as, isoprenaline, betahistine, scopolamine, and the like. Tranquilizers such as, reserprine, chlorpromazine, and antianxiety benzodiazepines such as, alprazolam, chlordiazepoxide, clorazeptate, halazepam, oxazepam, prazepam, clonazepam, flurazepam, triazolam, lorazepam, diazepam, and the like.

Antipsychotics such as, phenothiazines including thiopropazate, chlorpromazine, triflupromazine, mesoridazine, piperracetazine, thioridazine, acetophenazine, fluphenazine, perphenazine, trifluoperazine, and other major tranqulizers such as, chlorprathixene, thiothixene, haloperidol, bromperidol, loxapine, and molindone, as well as, those agents used at lower doses in the treatment of nausea, vomiting, and the like.

Drugs for Parkinson's disease, spasticity, and acute muscle spasms such as levodopa, carbidopa, amantadine, apomorphine, bromocriptine, selegiline (deprenyl), trihexyphenidyl hydrochloride, benztropine mesylate, procyclidine hydrochloride, baclofen, diazepam, dantrolene, and the like. Respiratory agents such as, codeine, ephedrine, isoproterenol, dextromethorphan, orciprenaline, ipratropium bromide, cromglycic acid, and the like. Non-steroidal hormones or antihormones such as, corticotropin, oxytocin, vasopressin, salivary hormone, thyroid hormone, adrenal hormone, kallikrein, insulin, oxendolone, and the like.

Vitamins such as, vitamins A, B, C, D, E and K and derivatives thereof, calciferols, mecobalamin, and the like for dermatologically use. Enzymes such as, lysozyme, urokinaze, and the like. Herb medicines or crude extracts such as, Aloe vera, and the like.

Antitumor agents such as, 5-fluorouracil and derivatives thereof, krestin, picibanil, ancitabine, cytarabine, and the like. Anti-estrogen or anti-hormone agents such as, tamoxifen or human chorionic gonadotropin, and the like. Miotics such as pilocarpine, and the like.

Cholinergic agonists such as, choline, acetylcholine, methacholine, carbachol, bethanechol, pilocarpine, muscarine, arecoline, and the like. Antimuscarinic or muscarinic cholinergic blocking agents such as, atropine, scopolamine, homatropine, methscopolamine, homatropine methylbromide, methantheline, cyclopentolate, tropicamide, propantheline, anisotropine, dicyclomine, eucatropine, and the like.

Mydriatics such as, atropine, cyclopentolate, homatropine, scopolamine, tropicamide, eucatropine, hydroxyamphetamine, and the like. Psychic energizers such as 3-(2-aminopropy)indole, 3-(2-aminobutyl)indole, and the like.

Antidepressant drugs such as, isocarboxazid, phenelzine, tranylcypromine, imipramine, amitriptyline, trimipramine, doxepin, desipramine, nortriptyline, protriptyline, amoxapine, maprotiline, trazodone, and the like.

Anti-diabetics such as, insulin, and anticancer drugs such as, tamoxifen, methotrexate, and the like.

Anorectic drugs such as, dextroamphetamine, methamphetamine, phenylpropanolamine, fenfluramine, diethylpropion, mazindol, phentermine, and the like.

Anti-malarials such as, the 4-aminoquinolines, alphaminoquinolines, chloroquine, pyrimethamine, and the like.

Anti-ulcerative agents such as, misoprostol, omeprazole, enprostil, and the like. Antiulcer agents such as, allantoin, aldioxa, alcloxa, N-methylscopolamine methylsuflate, and the like. Antidiabetics such as insulin, and the like.

For use with vaccines, one or more antigens, such as, natural, heat-killer, inactivated, synthetic, peptides and even T cell epitopes (e.g., GADE, DAGE, MAGE, etc.) and the like.

The drugs mentioned above may be used in combination as required. Moreover, the above drugs may be used either in the free form or, if capable of forming salts, in the form of a salt with a suitable acid or base. If the drugs have a carboxyl group, their esters may be employed.

The acid mentioned above may be an organic acid, for example, methanesulfonic acid, lactic acid, tartaric acid, fumaric acid, maleic acid, acetic acid, or an inorganic acid, for example, hydrochloric acid, hydrobromic acid, phosphoric acid or sulfuric acid. The base may be an organic base, for example, ammonia, triethylamine, or an inorganic base, for example, sodium hydroxide or potassium hydroxide. The esters mentioned above may be alkyl esters, aryl esters, aralkyl esters, and the like.

The nanoshell composition of the present invention may be formulated into a composition in a neutral and/or salt form. Pharmaceutically acceptable salts, include the acid addition salts (formed with the free amino groups of the protein) and/or that are formed with inorganic acids such as, for example, hydrochloric and/or phosphoric acids, and/or such organic acids as acetic, oxalic, tartaric, mandelic, and/or the like. Salts formed with the free carboxyl groups may also be derived from inorganic bases such as, for example, sodium, potassium, ammonium, calcium, and/or ferric hydroxides, and/or such organic bases as isopropylamine, trimethylamine, histidine, procaine and/or the like.

The carrier may also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity may be maintained, for example, by the use of a coating, such as lecithin, by the maintenance of the required particle size in the case of dispersion and/or by the use of surfactants. The prevention of the action of microorganisms may be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions may be brought about by the use in the compositions of agents delaying absorption, for example, aluminum monostearate and/or gelatin.

When a drug different than an anesthetic agent is used the solvent selected is one in that the drug is soluble. In generally the polyhydric alcohol may be used as a solvent for a wide variety of drugs. Other useful solvents are those known to solubilize the drugs in question.

Bioactive Delivery.

The bioactive may also be administered, e.g., parenterally, intraperitoneally, intraspinally, intravenously, intramuscularly, intravaginally, subcutaneously, or intracerebrally. Dispersions may be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fingi. The carrier may be a solvent or dispersion medium containing, for example, water, ethanol, poly-ol (for example, glycerol, propylene glycol, and liquid polyethylene glycol, and the like), suitable mixtures thereof, and vegetable oils.

The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

Sterile injectable solutions may be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier that contains a basic dispersion medium and the required other ingredients from those enumerated above. In the case of sterile powders for the preparation of sterile injectable solutions, the methods of preparation may include vacuum drying, spray drying, spray freezing and freeze-drying that yields a powder of the active ingredient (i.e., the therapeutic compound) plus any additional desired ingredient from a previously sterile-filtered solution thereof.

The bioactive may be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied as will be known to the skilled artisan. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a subject.

Aqueous compositions of the present invention comprise an effective amount of the nanoparticle, nanofibril or nanoshell or chemical composition of the present invention dissolved and/or dispersed in a pharmaceutically acceptable carrier and/or aqueous medium. The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes. The preparation of an aqueous compositions that contain an effective amount of the nanoshell composition as an active component and/or ingredient will be known to those of skill in the art in light of the present disclosure. Typically, such compositions may be prepared as injectables, either as liquid solutions and/or suspensions; solid forms suitable for using to prepare solutions and/or suspensions upon the addition of a liquid prior to injection may also be prepared; and/or the preparations may also be emulsified.

Dosage Forms.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts may be prepared in water suitably mixed with a surfactant, such as hydroxypropylcellulose. Dispersions may also be prepared in glycerol, liquid polyethylene glycols, and/or mixtures thereof and/or in oils. Under ordinary conditions of storage and/or use, these preparations contain a preservative to prevent the growth of microorganisms.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and/or the required other ingredients from those enumerated above. In the case of sterile powders for the preparation of sterile injectable solutions, the preferred methods of preparation are vacuum-drying and/or freeze-drying techniques that yield a powder of the active ingredient plus any additional desired ingredient from a previously sterile-filtered solution thereof. The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like may also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media that may be employed will be known to those of skill in the art in light of the present disclosure. For example, one dosage could be dissolved in 1 ml of isotonic NaCl solution and/or either added to 1000 ml of hypodermoclysis fluid and/or injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation.

Additional formulations that are suitable for other modes of administration include vaginal suppositories and/or suppositories. A rectal suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

In one particular embodiment, the drug and/or active agent may be introduced within the nanofibril by removing the sacrificial template around or about which the nanoparticles of the present invention are formed. For example, the V₂O₅ active template may be removed or exhausted during the formation of the nanoparticle polymer such that nanogap gaps and/or nanoholes are formed therein and into which one or more active agents may be introduced. The drug and/or active agent may be introduced via passive or active transport. Examples of passive transport include, e.g., diffusion, concentration gradients, suction and the like. Active transport may include, e.g., electrophoresis, chemical pumps, pressure, electrical potentials and the like. Depending on the charge or conductivity of the polymer(s), the drug and/or active agent, doping of the polymer, the carrier and the like, the skilled artisan will be able to select, without undue experimentation, the best combinations of charge, chemical entity, size, exclusion, and the like to maximize not only the introduction of the agent, but also its release once delivered to a location.

The examples of pharmaceutical preparations described above are merely illustrative and not exhaustive; the nanoparticles of the present invention are amenable to most common pharmaceutical preparations.

### EXAMPLE 1

### Synthesis of Polyaniline Nanofibers by “Nanofiber Seeding”

The present invention is a simple “nanofiber seeding” method to synthesize bulk quantities of nanofibers of the electronic polymer polyaniline in one step without the need for large organic dopants, surfactants, and/or large amounts of insoluble templates (Coelfen, H.; Mann, S. Angew. Chem., Int. Ed. 2003, 42, 2350). Seeding a conventional chemical oxidative polymerization of aniline with even very small amounts of biological, inorganic, or organic nanofibers (usually <1%) dramatically changes the morphology of the resulting doped polyaniline powder from nonfibrillar (particulate) to almost exclusively nanofibers. These findings could have widespread applications in morphological control in all precipitation polymerization reactions.

Conventional chemical oxidative polymerization approaches to nanostructured electronic polymers include the use of insoluble solid templates such as zeolites (Wu, C. G.; Bein, T. Stud. Surf. Sci. Catal. 1994, 84, 2269), opals (Misoska, V.; Price, W.; Ralph, S.; Wallace, G. Synth. Met. 2001, 121, 1501), and controlled pore-size membranes (Cepak, V. M.; Martin, C. R. Chem. Mater. 1999, 11, 1363.), or soluble templates such as polymers (Simmons, M. R.; Chaloner, P. A.; Armes, S. P. Langmuir 1995, 11, 4222) and surfactants (Yu, L.; Lee, J.-I.; Shin, K.-W.; Park, C.-E.; Holze, R. J. Appl. Polym. Sci. 2003, 88, 1550). A “nontemplate” approach has also been described in which the use of large organic anions results in polyaniline nanofibers and nanotubes having average diameters in the 650-80 nm range (Wan, M.; Wei, Z.; Zhang, Z.; Zhang, L.; Huang, K.; Yang, Y. Synth. Met. 2003, 135-136, 175). Recently, an interfacial polymerization method has been reported where 50 nm diameter fibers of polyaniline are produced at the interface of two immiscible liquids (Huang, J.; Virji, S.; Weiller, B. H.; Kaner, R. B. J. Am. Chem. Soc. 2003, 125, 314). Despite the diversity in these synthetic approaches, the dramatic change in polymer morphology points to an underlying mechanistic rationale; that is, polymeric nanostructures formed (or present) during the very early stages of the reaction can orchestrate bulk formation of similar nanostructures. This example shows that seeding a polymerization reaction with very small amounts of nanofibers, regardless of their chemical nature, results in a precipitate with bulk fibrillar morphology.

Seed nanofibers were chosen from a variety of organic, inorganic, and biological systems: (a) ˜50 nm diameter polyaniline nanofibers (as-synthesized HiPco SWNT) (Carbon Nanotechnologies, Inc), (b) ˜20 nm diameter single-walled carbon nanotube bundles (SWNT) made by the HiPco route (Von Bergen, M.; Friedhoff, P.; Biemat, J.; Heberle, J.; Mandelkow, E. M.; Mandelkow, E. Proc. Natl. Acad. Sci. U.S.A. 2000, 97, 5129), (c) ˜12 nm diameter nanofibrous hexapeptide, AcPHF6 (Ac-VQIVYK-amide), itself a seed in the polymerization of Alzheimer's disease tau protein (Bailey, J. K.; Pozarnsky, G. A.; Mecartney, M. L. J. Mater. Res. 1992, 7, 2530), and (d) ˜15 nm diameter nanofibers of V₂O₅.

Synthesis of Emeraldine.HCl Nanofibers. In a typical synthesis, to 60 ml of a stirred 0.14M solution of aniline in aq. 1.0M HCl, was added ˜1-4 mg of nanofibers of the seed template. To this mixture was added 40 ml of a 0.04M solution of the oxidant ammonium peroxydisulfate, also in aqueous 1.0M HCl. After 1.5 h, the resulting dark-green precipitate of emeraldine.HCl was suction filtered, washed with copious amounts of aqueous 1.0M HCl, and dried under dynamic vacuum at 80° C. for 12 h. The yield of emeraldine.HCl powder obtained was ˜200 mg.

The SEM images of the polyaniline powder obtained in all seeded experiments show fibrillar morphology with fibers having an average diameter in the range 20-60 nm (FIG. 1). It is important to note that just 1-4 mg of the seed nanofibers was sufficient to change the morphology of the bulk precipitate (˜200 mg) quantitatively to nanofibers (also confirmed by TEM). Unseeded reactions (conventional synthesis) or reactions seeded with particulate polyaniline powder yielded emeraldine•HCl precipitate having nonfibrous, particulate morphology. When the reaction was seeded with nanospheres of polypyrrole•Cl (˜50 nm diameter), however, the emeraldine•HCl precipitate was also largely in the form of nanospheres (˜170 nm diameter). Although the general shape of the seed appears to control the overall morphology of the precipitate (fibers versus particles), specific differences in the length, diameter, etc., of the seeds do not appear to have a significant impact.

FIG. 1A through 1D are SEM images of emeraldine•HCl nanofibers synthesized by seeding the reaction using the following: (Figure A) 1.5 mg of emeraldine.HCl nanofibers (SEM image inset), (FIG. 1B) 1.6 mg of HiPco SWNT (SEM image inset), (FIG. 1C) 1.0 mg of the hexapeptide AcPHF6 (TEM image inset), and (FIG. 1D) 4 mg of V₂O₅ nanofibers (SEM image inset).

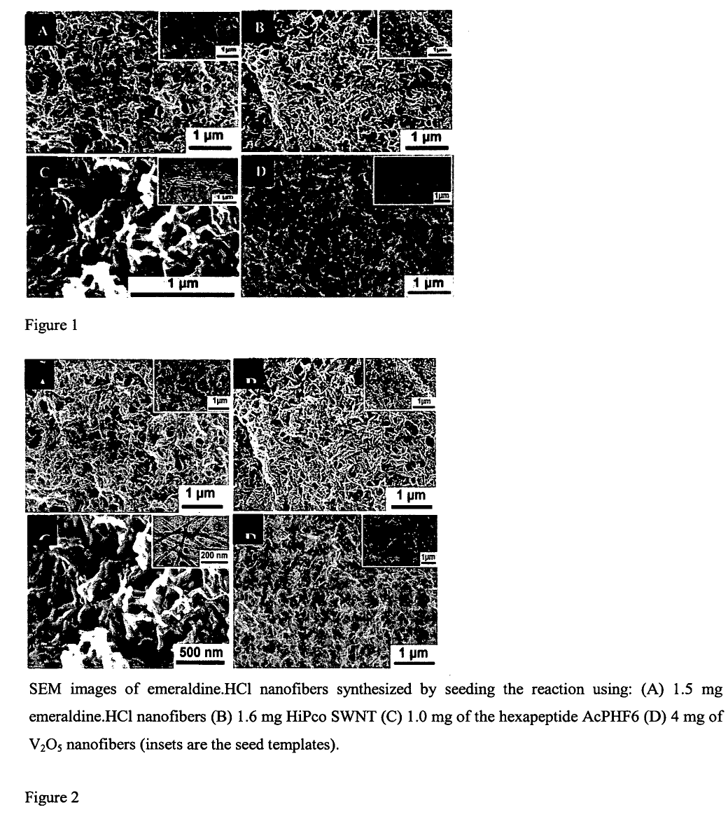

FIGS. 2A to 2D are high magnification images of emeraldine•HCl nanofibers by seeding the reaction with: (FIG. 2A) 1.5 mg emeraldine•HCl nanofibers (SEM images); (FIG. 2B) 1.6 mg HiPco SWNT (SEM images); (FIG. 2C) 1.0 mg hexapeptide AcPHF6 (TEM images); and (FIG. 2D) 4 mg V₂O₅ nanofibers (SEM images).

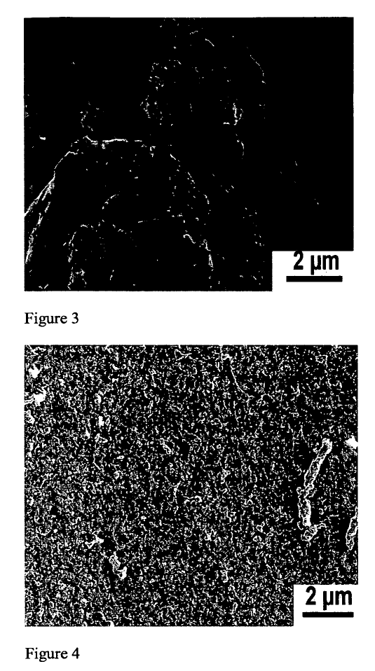

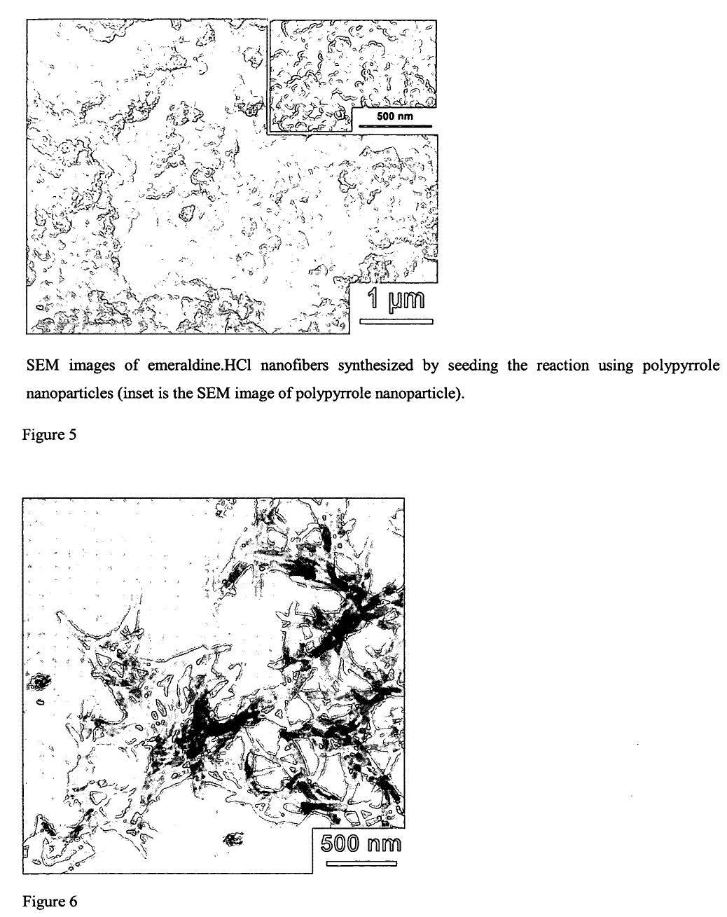

FIG. 3 is an SEM image of emeraldine•HCl nanofibers using an unseeded (conventional) chemical polymerization. FIG. 4 is an SEM image of emeraldine•HCl powder on glass slides synthesized using an unseeded (conventional) chemical polymerization. FIG. 5 is an SEM image of nanoparticles of emeraldine•HCl nanospheres synthesized using a seeded reaction of doped polypyrrole•Cl using FeCl₃ as an oxidant. FIG. 6 is a TEM image of emeraldine•HCl nanofibers using a seeded reaction using SWNT.

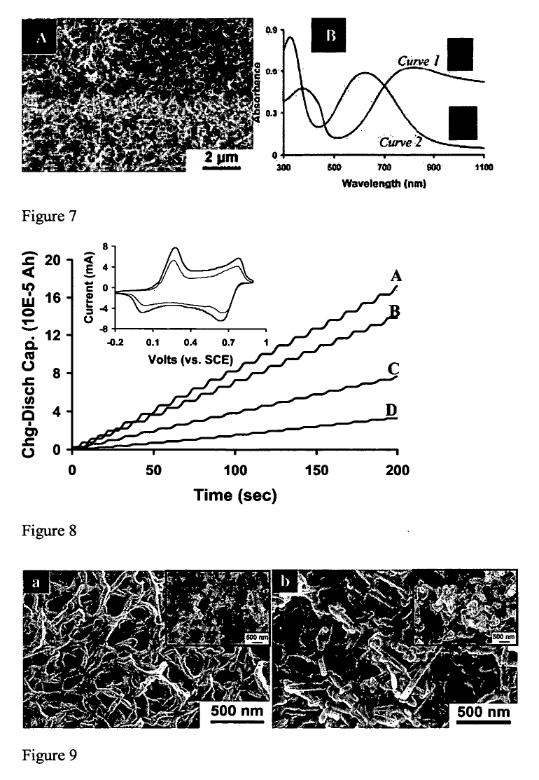

FIG. 7A is an SEM image (left) of an in-situ deposited film of emeraldine•HCl nanofibers on a glass microscope slide synthesized using 1.6 mg of SWNT seed template.

FIG. 7B is a graph of the solid-state UV/vis spectra and optical images of films of emeraldine•HCl (green, curve 1) and emeraldine base (blue, curve 2) on a glass microscope slide. During the synthesis, the walls of the reaction flask were also coated with a dark-green film of in-situ deposited emeraldine•HCl. This film, normally observed during conventional chemical oxidative polymerization of aniline, has been extensively investigated in the past (Ayad, M. M.; Gemaey, A. H.; Salahuddin, N.; Shenashin, M. A. J. Colloid Interface Sci. 2003, 263, 196; Sapurina, I.; Riede, A.; Stejskal, J. Synth. Met. 2001, 123, 503). In SWNT seeded systems, this in-situ deposited film also has a nanofibrillar morphology (FIG. 7A). These in-situ deposited films are thin (<1 μm), transparent, and strongly adherent, which permits their facile and rapid characterization without requiring cumbersome postsynthesis processing steps, for example, product isolation, spin coating, etc. This approach is useful for use in a variety of technological applications requiring the use of substrate-supported film, for example, sensors, displays, etc.

Spectroscopically, both powders and films of polyaniline nanofibers were essentially identical to conventional nonfibrillar emeraldine•HCl (MacDiarmid, A. G. ReV. Mod. Phys. 2001, 73, 701). For example, the FT/IR (KBr pellet) and solution UV/vis (in NMP) spectra of the corresponding base forms are consistent with the polymer being in the emeraldine oxidation state (Albuquerque, J. E.; Mattoso, L. H. C.; Balogh, D. T.; Faira, R. M.; Masters, J. G.; MacDiarmid, A. G. Synth. Met. 2000, 113, 19), and the UV/vis spectra of in-situ deposited films of the emeraldine•HCl and emeraldine base nanofibers on glass microscope slides (FIG. 7B) are also similar to those obtained previously. Four-probe pressed-pellet conductivities for polyaniline nanofibers were in the range 2-10 S/cm, similar to conventional emeraldine•HCl powder. There is also no significant difference in their aqueous electrochemistry; that is, the cyclic voltammogram of emeraldine, HCl nanofibers displays the two redox peaks characteristic of parent polyaniline (MacDiarmid, A. G.; Yang, L. S.; Huang, W.-S.; Humphrey, B. D. Synth. Met. 1987, 18, 393) (inset, FIG. 8).

FIG. 8 is a graph that shows the charge/discharge capacity plot of emeraldine.HCl powder in the range 0.4-0.5 V (vs SCE) in aqueous 1.0 M camphorsulfonic acid electrolyte. Charge (curve A), discharge (curve B) cycles for nanofibers and charge (curve C), discharge (curve D) cycles for conventional (nonfibrillar) polyaniline. Inset: cyclic voltammograms of polyaniline nanofibers (outer plot) and conventional polyaniline (inner plot). There is, however, a significant difference in the capacitance values for polyaniline nanofibers. For example, a capacitance value of 122 F/g was obtained for emeraldine.HCl nanofibers synthesized using polyaniline (nanofibers) as the seed template as compared to 33 F/g in nonfibrillar emeraldine•HCl (FIG. 8). Elevated capacitance values were obtained for all seeded systems. The voltage range 0.4-0.5 V (vs SCE) was chosen because it falls in the valley between the two redox peaks of polyaniline (see inset in FIG. 8). The charge/discharge cycles are also more symmetrical in a nanofiber (FIG. 8, curves A, B), which is consistent with their increased available surface area that is expected to improve the kinetics of the various processes involved, and could play an important role in the development of next-generation energy storage devices.

The reasons for the fibrillar morphology in all seeded systems are not clear, although this morphology could be related to fibrillar morphology observed in the electrochemical polymerization of aniline in the presence of the aniline dimer, N-phenyl-1,4-phenylenediamine (Wei, Y.; Sun, Y.; Jang, G.-W.; Tang, X. J. Polym. Sci., Part C: Polym. Lett. 1990, 28, 81). Two important factors common to this class of precipitation polymerization reactions are as follows: (i) there is an induction period followed by a rather rapid formation of a precipitate, and (ii) the influence of inert surfaces (walls of the reaction flask, etc.) on progress of the reaction. Polymerization first occurs on the surface of the seed template whose morphology is mirrored by the growing polymer chain. Indeed, a blue-green film of pernigraniline salt is formed on the walls of the reaction flask, magnetic stir bar, etc., well before any precipitate is observed in bulk. The in-situ deposited film of (fibrillar) pernigraniline salt can then seed fresh polymer growth triggering a continuous seeding process resulting in a bulk precipitate in which the nanoscale morphology of the original seed template is transcribed over many length scales. This phenomenon can also be extended to other electronic polymers, for example, polypyrrole and PEDOT.

The effect that even small amounts of insoluble substances can have on the properties of the final product is surprising and raises important questions and concerns in the area of precipitation polymerization in general and synthesis of electronic polymers in particular. For example, during the chemical or electrochemical synthesis of electronic polymers, special care must taken to ensure that the reaction system is free of particulate matter like inadventitious dust, fabric lint, etc. It is perhaps not surprising that one can find in the literature several examples of polyaniline synthesized using “established procedure” but exhibiting very different properties, raising questions that have been consistently voiced by the scientific community (Stejskal, J.; Gilbert, R. G. Pure Appl. Chem. 2002, 74, 857).

In summary, this example demonstrates the development of emergent nanostructures in electronic polymers over multiple length scales triggered by very small amounts of added nanoscale templates. Described for the first time are the following: (i) the use of nanostructured seed templates to synthesize rapidly, and in one step, bulk quantities of doped polyaniline nanofibers without the need for conventional templates, surfactants, polymers, or organic solvents; (ii) a convenient method to obtain thin, substrate-supported, transparent films of nanofibers of polyaniline without requiring any bulk processing steps; (iii) increased capacitance values in polyaniline nanofibers synthesized by the nanofiber seeding method; and (iv) a general phenomenon impacting the field of precipitation polymerizations that could facilitate the design of next-generation electronic polymer systems requiring nanometer scale control of surface architecture.

### EXAMPLE 2

### Chemical Synthesis of Polyaniline Nanofibers using Surfactants

Nanofibers of doped polyaniline•HCSA having diameters 1-2 nm are observed in TEM images of bath sonicated aqueous dispersions of larger nanofibers (30-50 nm diameter) synthesized by surfactant-assisted chemical oxidative polymerization of aniline in dilute aqueous organic acids.

The present example describes a simple and rapid one-phase surfactant-assisted chemical method to synthesize bulk quantities of analytically pure nanofibers of polyaniline doped with d,l-camphorsulfonic acid (emeraldine•HCSA) and with 2-acrylamido-2-methyl-1-propanesulfonic acid (emeraldine-AMPSA). A conventional chemical oxidative polymerization of aniline in 1.0M HCSA or AMPSA using ammonium peroxydisulfate oxidant, when carried out in the presence of added non-ionic surfactant Triton-X 100 (TX100) results in a precipitate of doped emeraldine salt composed almost entirely of nanofibers having average fiber diameter in the range 30-50 nm and exhibiting a room temperature DC conductivity of 1-5 S/cm. Fiber diameter can be driven even lower by bath sonication to yield a single molecule fiber of emeraldine•HCSA (1-2 nm diameter) as shown by TEM.

While polyaniline with fibrillar morphology has been chemically synthesized using insoluble (hard) templates (H. Qiu, J. Zhai, S. Li, L. Jiang and M. Wan, Adv. Funct. Mater. 2003, 13, 925; Z. Wei, M. Wan, T. Lin and L. Dai, Adv. Mater. 2003, 15, 136; R. V. Parthasarathy and C. R. Martin, Chem. Mater. 1994, 6, 1627; C. G. Wu and T. Bein, Stud. Surf. Sci. Catal. 1994, 84, 2269.), soluble (soft) templates (L. Zhang and M. Wan, Nanotechnology 2002, 13, 750; M. Wan, Z. Wei, Z. Zhang, L. Zhang, K. Huang and Y. Yang, Synth. Met. 2003, 135-136, 175), pseudo-templates like large organic dopant anions (Z. Wei and M. Wan, J. Appl. Polym. Sci. 2003, 87, 1297; Z. Wei, Z. Zhang and M. Wan, Langmuir 2002, 18, 917), and more recently, by interfacial polymerization (J. Huang and R. B. Kaner, J. Am. Chem. Soc. 2004, 126, 851; S. Virji, J. Huang, R. B. Kaner and B. H. Weiller, Nano Lett. 2004, 4, 491), the use of surfactants during the polymerization, i.e., micellar and emulsion polymerization systems has largely yielded polyaniline having particulate (non-fibrillar) morphology (J. Stejskal, M. Omastova, S. Fedorova, J. Prokes and M. Trchova, Polymer 2003, 44, 1353; T. Jana and A. K. Nandi, J. Mater. Res. 2003, 18, 1691; M. G. Han, S. K. Cho, S. G. Oh and S. S. Im, Synth. Met. 2002, 126, 53; D. Kim, J. Choi, J.-Y. Kim, Y.-K. Han and D. Sohn, Macromolecules 2002, 35, 5314; W. Liu, J. Kumar, S. Tripathy and L. A. Samuelson, Langmuir 2002, 18, 9696). There are very few instances where fibrillar morphology has been observed in surfactant-assisted polymerization of aniline (L. Yu, J.-I. Lee, K.-W. Shin, C.-E. Park and R. Holze, J. Appl. Polym. Sci. 2003, 88, 1550; J.-E. Osterholm, Y. Cao, F. Klavetter and P. Smith, Polymer 1994, 35, 2902), and to the best of our knowledge, there has not been any report on the use of non-ionic surfactants to generate polyaniline having bulk nanofiber morphology. The present example describes: (i) the use of a combination of large organic dopants and non-ionic surfactants such as TX100 to synthesize highly conducting nanofibers of polyaniline, and (ii) attempts to drive down the fiber diameter closer to the one-dimensional (single molecule fiber) regime.

The polyaniline precipitate obtained by chemical oxidative polymerization of aniline in aqueous 1.0M HCSA or AMPSA in the presence of TX100 is composed almost entirely of nanofibers having average diameter in the range 30-50 nm. The insets in FIG. 9 describe the morphology of polyaniline obtained under identical conditions in the absence of TX100. Polyaniline nanofibers synthesized using TX100 for both HCSA and AMPSA systems are analytically and spectroscopically similar to corresponding samples synthesized without TX100. The doping percentage, calculated from elemental analyses (sulfur/nitrogen ratio) was 43% for emeraldine.HCSA (FIG. 9A) and 45% for emraldine.AMPSA (FIG. 9B). The elemental analyses also showed slightly elevated oxygen levels which persist even upon several doping/dedoping cycles and extended drying under dynamic vacuum at 80° C., suggesting its origin to water of hydration, or to water trapped inside the fiber should the fibers be hollow. For both systems, vibrational spectra (KBr pellet), cyclic voltammetry (aqueous 1.0M HCl versus SCE) and pressed pellet 4-probe room temperature conductivity values (1-5 S/cm) are essentially identical to the corresponding emeraldine salts synthesized without TX100. Expectedly, significantly higher capacitance values are obtained for polyaniline nanofibers synthesized using TX100 which is consistent with its high surface area.

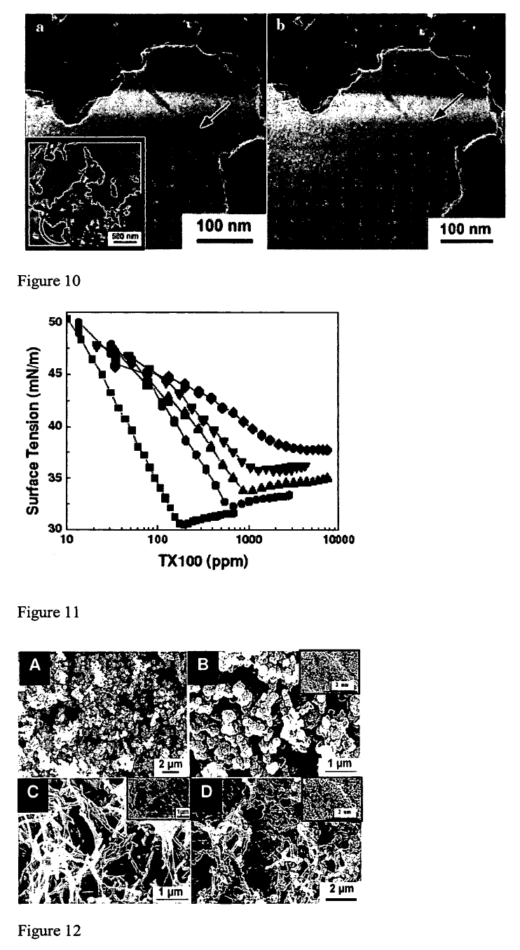

Polyaniline nanofibers obtained in this study are chemically robust and retain their fibrillar morphology even after repeated doping and dedoping cycles using aqueous acids and bases, although they deform readily under mechanical stress and fragment to smaller pieces under strong probe sonication. TEM images obtained after moderate mechanical agitation, e.g., bath sonication for 2 h in water show very small diameter nanofibers (1-2 nm) distributed among fragmented clusters of the original larger nanofibers (FIG. 10A, inset). An expanded section of this image (FIG. 10A) shows a thin, 2-5 nm fiber bridging two regions of fragmented fiber clusters. At the center of the bridge, over a length of 40 nm, the fiber appears to become so thin that it's image does not register which is consistent with a fiber having diameter in 1-2 nm range (instrument limit). When the electron beam was focused on this area (see arrow in FIGS. 10A and 10B), the fiber begins to vibrate and then breaks cleanly into two independently vibrating fibers (FIG. 10B, was also seen in video imaging) confirming the presence of a very thin fiber in this region. Molecular models and crystal structure studies of emeraldine•HCSA show that the ‘diameter’ of a single chain is in the range 1.0-1.8 nm (W. Luzny and E. Banka, Macromolecules 2000, 33, 425) suggesting that the TEM image, in this region of the sample, is consistent with that of a single molecule fiber of doped polyaniline. Size exclusion chromatography of the correponding emeraldine base powder in NMP/LiBF₄ eluent (60° C./polystyrene standards) shows a unimodal gaussian peak and Mw 20,000 (PD 2.2) indicating that the chains are long enough to form 40 nm long fibers. A close observation of the TEM images reveals that these very small diameter nanofibers are present in all parts of the sample and may even be present in emeraldine•HCSA reported in previous studies (J. Huang and R. B. Kaner, J. Am. Chem. Soc. 2004, 126, 851; S. Virji, J. Huang, R. B. Kaner and B. H. Weiller, Nano Lett. 2004, 4, 491). Alternatively, if gentle bath sonication is in some way responsible for the formation of these very small diameter fibers from larger fibers, this method could be an attractive post synthesis ‘processing step’ to synthesize smaller diameter fibers in larger quantities.

The role of TX100 in promoting fibrillar polymer growth is not clear, e.g., a close examination of SEM images of polyaniline synthesized without TX100 (FIG. 9A, 9B insets) show chemical oxidative polymerization of aniline using ammonium peroxydisulfate oxidant in aqueous solution of 1.0 M organic acids (HCSA, AMPSA) when carried out in the presence of non-ionic surfactants such as Triton-X100, results in a polyaniline precipitate having bulk nano-fibrillar morphology (30-50 nm diameter). The surfactant solution microstructure plays an important role in fiber formation with best fibers observed above the composite critical micelle concentration. Gentle bath sonication of these nanofibers results in 1-2 nm diameter fibers of emeraldine•HCSA of a single-molecule fiber of a doped conducting polymer.

There may be a connection between the critical micelle concentration (CMC) of TX100 in the reaction mixture and fiber formation. Longer, more uniformly distributed and smaller diameter fibers are produced at TX100 concentrations in the range 2,500-4,000 ppm for the HCSA system and 800-1,200 ppm for the AMPSA system. The typically low CMC values observed in aqueous TX100 solutions (100-200 ppm in inorganic acids)(S. Ouni, A. Hafiane and M. Dhahbi, C. R. Acad. Sci. Paris 2000, 3, 353; R. Sharma, D. Varade and P. Bahadur, J. Dispersion Sci. Technol. 2003, 24, 53) increases significantly to 1,100 ppm in 1.0M HCSA and 620 ppm in 1.0M AMPSA. When aniline is added the CMC increases even further, i.e., to 2,200 ppm (HCSA system) and 866 ppm (AMPSA system). The initial increase in CMC is caused presumably by mixed micelle formation and/or incorporation of these large organic anions in Stern layer of the micelle. The subsequent increase in CMC is consistent with cation exchange between protons and anilinium ions at the micelle water interface. There is also a significant increase in surface tension consistent with charge buildup in the micellar aggregate from the negatively charged sulfonate headgroup. Best nanofibers are obtained above the composite CMC of the system suggesting that micelle-water interface is playing an important role (S. Ouni, A. Hafiane and M. Dhahbi, C. R. Acad. Sci. Paris 2000, 3, 353; R. Sharma, D. Varade and P. Bahadur, J. Dispersion Sci. Technol. 2003, 24, 53).

It is important to note that unlike typical aniline polymerization reactions, these reactions were not stirred or mechanically agitated in any way. Polymerization is expected to be initiated at the micelle-water interface because of the increased local aniline concentration and since our system is not agitated, aniline dimer and higher oligomers are expected to accumulate at the micelle-water interface. These dimers and oligomers may be responsible for orchestrating fibrillar polymer growth. This is consistent with nanofibrillar morphology previously observed in chemical and electrochemical polymerization of aniline in the presence of added aniline oligomers (L. Duic, M. Kraljic and S. Grigic, J. Polym. Sci., Part A: Polym. Chem. 2004, 42, 1599; W. Li and H.-L. Wang, J. Am. Chem. Soc. 2004, 126, 2278; Y. Wei, Y. Sun, G. W. Jang and X. Tang, J. Polym. Sci., Part C: Polym. Lett. 1990, 28, 81; C. Mailhe-Randolph and A. J. McEvoy, Ber. Bunsen-Ges. Phys. Chem 1989, 93, 905.). Precisely how aniline oligomers promote fibrillar polymer growth is unclear, although we believe that the nascent polyaniline precipitate formed during the early stages of the reaction must also possess fibrillar morphology. Since aniline polymerization has been shown to be autocatalytic with the loci of polymerization shifting from bulk solution to the nascent oligomeric chains, the morphology of these ‘seed’ oligomers is transcribed to the bulk precipitate. This is analogous to our recently reported ‘nanofiber seeding’ synthesis of polyaniline (X. Zhang, W. J. Goux and S. K. Manohar, J. Am. Chem. Soc. 2004, 126, 4502).

In summary, this example demonstrates: (i) the use of nonionic surfactants to synthesize rapidly, and in one step, bulk quantities of doped polyaniline nanofibers without the need for conventional templates, polymers or organic solvents, (ii) unusually high composite CMC values for TX100 in organic acid/aniline systems and its role in orchestrating bulk nanofibrillar morphology, and (iii) a simple method to ‘process’ larger polyaniline nanofibers into smaller, 1-2 nm fibers in what we believe is the first report of a single molecule fiber of a doped conducting polymer.

### EXAMPLE 3

### Polypyrrole Nanofibers: A Direct Chemical Synthetic Route

The present example is a direct, one-step bulk chemical synthetic route to nanofibers of the electronic organic polymer polypyrrole using a variant of the nanofiber seeding method (described hereinabove) for synthesizing bulk quantities of nanofibers of polyaniline (Zhang, X.; Goux, W. J.; Manohar, S. K. J. Am. Chem. Soc. 2004, 126, 4502). Using the method described herein, a key synthetic challenge in the control of nanostructure of electronic polymers (beyond polyaniline) has been met by uncovering an important chemical property for seed templates to effectively orchestrate fibrillar polymer growth, i.e., the seed template must itself be capable of oxidatively reacting with the monomer (pyrrole, EDOT, etc.).

Polypyrrole is a technologically important, environmentally stable conducting polymer exhibiting high electronic conductivity at physiological pH (Lee, E. S.; Park, J. H.; Wallace, G. G.; Bae, Y. H. Polym. Int. 2004, 53, 400). While there are several reports describing the synthesis of polypyrrole fibers within the pores of templates such as zeolites (Ikegame, M.; Tajima, K.; Aida, T. Angew. Chem. Int. Ed. 2003, 42, 2154), alumina (Li, X.; Zhang, X.; Li, H. J. Appl. Polym. Sci. 2001, 81, 3002. He, J.; Chen, W.; Xu, N.; Li, L.; Li, X.; Xue, G. Appl. Surf. Sci. 2004, 221, 87) and particle track-etched membrane (Cai, Z.; Martin, C. R. J. Am. Chem. Soc. 1989, 111, 4138. Duvail, J. L.; Retho, P.; Godon, C.; Marhic, C.; Louam, G.; Chauvet, O.; Cuenot, S.; Nysten, B.; Dauginet-De Pra, L.; Demoustier-Champagne, S. Synth. Met. 2003, 135-136, 329. Duchet, J.; Legras, R.; Demoustier-Champagne, S. Synth. Met. 1998, 98, 113. Pyo, M.; Cho, C. J. Appl. Polym. Sci. 2002, 85, 514. Ermolaev, S. V.; Jitariouk, N.; Le Moel, A. Nuc. Instr. Meth. B: 2001, 185, 184. De Vito, S.; Martin, C. R. Chem. Mater. 1998, 10, 1738), etc., the bulk synthesis of nanofibers of polypyrrole directly from pyrrole monomer, i.e., with average fiber diameter <100 nm, has been a challenge. Approaches such as surfactant-mediated synthesis (Li, G.; Zhang, Z. Macromolecules, 2004, 37, 2683), interfacial synthesis (Huang, J.; Kaner, R. B. J. Am. Chem. Soc. 2004, 126, 851), and nanofiber seeding (Zhang, supra), etc., that have been so successful in the synthesis of nanofibers of polyaniline yield only non-fibrous, granular powders in the case of polypyrrole. Fibrillar and tubular morphology in polypyrrole has been observed when large organic dopant anions such as naphthalenesulfonic acid are used during the synthesis (Shen, Y.; Wan, M. J. Polym. Sci. Part A: 1999, 37, 1443). These fibers and tubes have relatively large diameters (>400 nm) and are formed presumably as a result of the solution aggregation of the dopant anions. This example shows a new approach to nanofiber formation, namely, the use of reactive seed templates that chemically react with the monomer prior to the addition of oxidant. The pre-polymerization reaction on the surface of fibrillar seed templates helps direct the evolution of bulk fibrillar morphology when oxidant is subsequently added. No fibrillar morphology is observed when passive, or inert seed templates are used.

The morphology of doped polypyrrole•Cl powder changes dramatically from granular to nanofibrillar when very small amount (1-4 mg) of V2O5 nanofibers are added to a chemical oxidative polymerization of pyrrole in aqueous 1.0M HCl using (NH₄)₂S₂O₈ as the oxidant. Unlike the polyaniline system, a key synthetic requirement in the polypyrrole system is for the seed template to be ‘active’, i.e., to be capable of independently oxidizing the pyrrole monomer. Thin, strongly adherent films can be obtained on inert surfaces such as glass, plastics, etc., directly from the polymerization mixture without any bulk product isolation steps, significantly simplifying the processing of these nanofibers.

Synthesis of Polypyrrole.Cl Nanofibers. In a typical experiment, to 60 ml of a stirred 0.24M solution of pyrrole in aqueous 1.0M HCl, was added ˜1-4 mg of nanofibers of the seed template. To this mixture was added 20 ml of a 0.22M solution of the oxidant ammonium peroxydisulfate, also in aqueous 1.0M HCl. After 20 min, the resulting dark precipitate of Polypyrrole.Cl was suction filtered, washed with copious amounts of aq. 11.0M HCl, and dried under dynamic vacuum at 80° C. for 12 h. The yield of Polypyrrole.Cl powder obtained was ˜300 mg.

Granular polypyrrole.Cl is obtained in unseeded reactions (FIG. 12A) or other control reactions seeded by inert seed templates, e.g., 30-50 nm diameter polypyrrole nanofibers, or 20-30 nm diameter HiPco single-walled carbon nanotube bundles (SWNT) (Zhang, supra) (FIG. 12B). Seeding the reaction with 1-4 mg of 15 nm diameter nanofibers of V₂O₅ (Bailey, J. K.; Pozarnsky, G. A.; Mecartney, M. L. J. Mater. Res. 1992, 7, 2530), however, dramatically alters the bulk morphology of the product to almost exclusively nanofibers (FIG. 12C). The elemental analysis of the nanofibers is summarized in Table 1. Briefly, the elemental analysis is as follows: C, 56.91; H, 3.97; N, 16.53; O: 9.27; Cl: 15.53; S: 0.0; V: 0.0, is consistent with the structure (PPy)(Cl)_{0.37}(H₂O)_{0.49}. These fibers are highly conducting (σ_{RT}˜50 S/cm), analytically pure, and free of any HSO₄ co-dopant (normally present in the product when V₂O₅ is not used). The elemental analysis also shows that there is no residual V in the product indicating that the V₂O₅ seed template is quantitatively removed without the need for additional template removal steps.

TABLE 1
Elemental analysis of Polypyrrole.Cl synthesized
in the presence of V₂O₅ “nanofiber seed”.

| Elements | Theory | Found | H2O |
|---|---|---|---|
| C | 55.41 | 56.91 | 0 |
| H | 4.21 | 3.97 | 1.16 |
| N | 16.16 | 16.53 | 0 |
| O | 9.05 | 9.27 | 9.27 |
| Cl | 15.17 | 15.53 | 0 |
| V | 0 | 0 | |
| total | 100.00 | 102.21 | |

PPy/Cl(0.37)/H₂O (0.49)

The solution darkened noticeably when the V₂O₅ seed was added to the pyrrole/HCl solution prior to the addition of (NH₄)₂S₂O₈, consistent with the oxidation of pyrrole monomer on the surface of V₂O₅. Subsequent addition of (NH₄)₂S₂O₈ resulted in the rapid precipitation of polypyrrole.Cl powder having bulk nanoscale morphology. The analogous control reaction seeded with granular V₂O₅ also darkened in color, but yielded only granular polypyrrole. In contrast, there was no darkening of the reaction solution when SWNT or other inert seeds were used. Therefore, nanofibrillar morphology was observed in systems in which the seed template must: (i) itself possess nanofibrillar morphology, and (ii) also be capable of oxidatively reacting with the monomer.

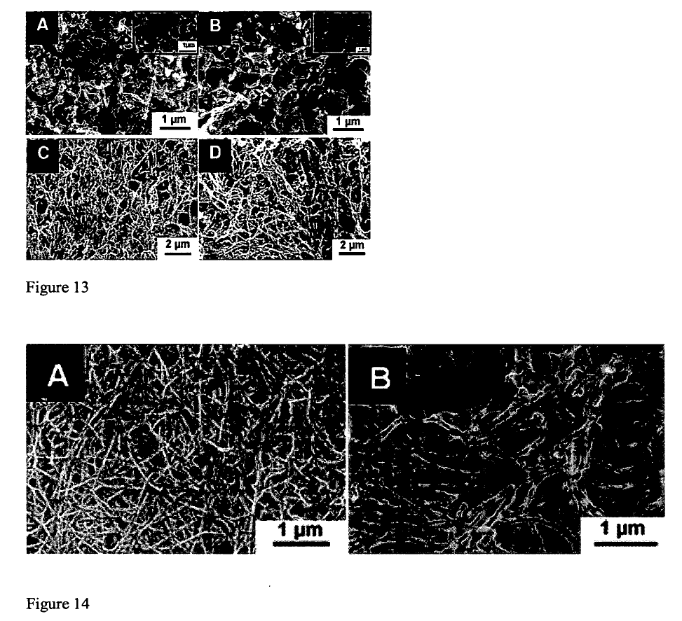

The need for the seed template to also be chemically reactive towards the monomer is borne out in SWNT-seeded reactions in which the SWNT was pre-exposed to (NH₄)₂S₂O₈ for 20 min before pyrrole monomer was added. In contrast to the ‘inert’ SWNT seeded reaction (FIG. 12B), reactions seeded by SWNT pre-exposed to (NH₄)₂S₂O₈ resulted in polypyrrole.Cl having nanofibrillar morphology (FIG. 12D). It was possible that polymerization is initiated by (NH₄)₂S₂O₈ adsorbed on the SWNT surface in which the nanoscale morphology of the now ‘active’ seed is transcribed across several length scales to the bulk precipitate. Additional evidence for the need for the seed template to be ‘activated’ was obtained in cross-seeding studies using polyaniline as the seed template. For example, granular polypyrrole.Cl is obtained when the reaction is seeded with polyaniline nanofibers in the emeraldine oxidation state (inert seed) while a significantly larger amount of nanofibrillar polypyrrole.Cl is obtained when polyaniline nanofibers in the pernigraniline oxidation state (oxidized) is used instead (FIG. 13A). The oxidation potential of the pernigraniline oxidation state (V_{oc}0.8V vs. SCE)(Manohar, S. K.; MacDiarmid, A. G.; Epstein, A. J. Synth. Met. 1991, 41, 711) is sufficiently high to initiate the oxidation of pyrrole monomer (0.5V versus SCE). Smooth, uniform polypyrrole.Cl nanofibers are obtained when the reaction is seeded by nanofibers of the ‘polyaniline’ in the pernigraniline oxidation state synthesized by the chemical oxidative polymerization of the aniline dimer, N-phenyl-1,4-phenylenedamine (FIG. 13B).

Normally, there are significant challenges in processing chemically synthesized polypyrrole.Cl into films, fibers, etc., since it is intractable and insoluble in common organic solvents. However, thin, strongly adherent and transparent substrate-supported films on glass, plastics, etc., are readily obtained directly from the synthesis without even having to isolate the bulk product (FIGS. 13C, 13D). These films, formed by electroless deposition of polypyrrole on inert surfaces present during the reaction, have been investigated extensively in the past under the umbrella of in-situ adsorption polymerization (Ayad, M. M. J. Mater. Sci. Lett. 2003, 22, 1577). The morphology of these films nicely mirrors the bulk powder, permitting the rapid and facile characterization of these nanofibers and their use in the fabrication of plastic electronic devices and sensors.

Fiber diameter can be controlled when the reaction is carried out in ethanol using FeCl₃ as the oxidant and by pre-exposing the V₂O₅ seed template to ethanol before the reaction. Thinnest fibers (30 nm) were obtained by stirring the V₂O₅ seed template in ethanol for 30 min and fiber thickness increases to 100 nm upon stirring for 12 hours (FIG. 14B). The increase in the thickness of the fibers may be due to, e.g., bundling of V₂O₅ nanofibers into thicker fibers upon extended stirring in ethanol. The polypyrrole fibers obtained with ethanol as the solvent are smoother, more uniform and free of any granular product compared to polypyrrole synthesized under aqueous conditions.

The need to ‘activate’ the seed template to ensure fibrillar polymer growth in polypyrrole vs. polyaniline is presumably due to fundamental differences between the two systems. Unlike the polypyrrole system, a small amount of nanofibers are observed even in the unseeded polyaniline system, suggesting that fibrillar polymer growth is intrinsic to polyaniline, and the added seed template directs the synthetic trajectory along these pre-existing pathways. In the polypyrrole system, however, these pathways would have to be induced, e.g., by using seed templates that are either intrinsically reactive towards pyrrole monomer (V₂O₅), or those that can be rendered reactive by treatment with (NH₄)₂S₂O₈ (SWNT, polyaniline, aniline dimer). It is to be noted that the precise mechanism(s) responsible for the dramatic change in morphology in the presence of nanostructured seed templates (for both systems) remains to be elucidated.

In summary, this example demonstrates: (i) a rapid and convenient method to chemically synthesize bulk quantities of microns long, 60-90 nm thick nanofibers of electronically conducting polypyrrole directly from pyrrole; (ii) a convenient electro-less, room-temperture deposition method to process these nanofibers in the form of thin, strongly adherent coatings on a variety of substrates without any product isolation steps; (iii) control of fiber diameter by using non-aqueous solvents; and (iv) a new phenomenon, i.e., the use of reactive seed templates to induce bulk nanoscale morphology. These findings also have potential to be leveraged beyond conducting polymers to embrace the broad class of precipitation polymerization reactions, e.g., they can be used to induce nanoscale morphology in polymerization reactions that are intrinsically recalcitrant to fibrillar polymer growth.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of controlled polymer formation comprising the step of:
polymerizing in a single step:
a structural substrate;
an oxidatively reactive monomer; and
an oxidant to form a polymer that takes the morphology of the structural substrate.

2. The method of claim 1, wherein the structural substrate is defined further as an active substrate.

3. The method of claim 1, wherein the oxidatively reactive monomer polymerizes into a bio-compatible polymer.

4. The method of claim 1, wherein the oxidatively reactive monomer polymerizes into a bio-degradable polymer.

5. The method of claim 1, wherein the oxidatively reactive monomer polymerizes into a bio-degradable and bio-compatible polymer.

6. The method of claim 1, wherein the oxidatively reactive monomer is selected from lactic acid, glycolic-lactic acid, glycolic acid, cyanobutylacrylate, propylbutyl, polypyrrole, 3,4-ethylenedioxythiophene, pyrrole, aniline and combinations thereof.

7. The method of claim 1, wherein the polymerization uses one or more of the following polymerizable monomers: ethylene glycol; ethylene oxide; partially or fully hydrolyzed vinyl alcohol; vinylpyrrolidon; ethyloxazoline; ethylene oxide-co-propylene oxide; block copolymers, poloxamers, meroxapols, poloxamines; conductive polymers; methyl, laural, stearyl or butyl methacrylates; vinyl halides; tetrafluroethylene, acrylonitrile, ethylene; methyl, ethyl or butyl acrylates and combinations thereof.

8. The method of claim 1, wherein a second polymer is added to the polymerization reaction selected from natural polymers comprising carboxymethyl cellulose, and hydroxyalkylated celluloses such as hydroxyethyl cellulose and methylhydroxypropyl cellulose, polypeptides, polysaccharides or carbohydrates, polysucrose, hyaluronic acid, dextran, heparan sulfate, chondroitin sulfate, heparin, and alginate, and proteins such as gelatin, collagen, albumin, and ovalbumin, other copolymers, and combinations thereof.

9. The method of claim 1, further comprising the step of adding a bioactive compound.

10. The method of claim 8, wherein the bioactive compound is selected from the group consisting of a drug, a protein, a peptide, a polysaccharide, an oligonucleotide, an antibiotic, an anti-cancer drug, an antigen, an antibody, a bioactive extract, a synthetic organic molecule, and a synthetic inorganic molecule.

11. The method of claim 1, wherein the structural substrate comprises an inorganic molecule, a crystal, a magnet, a metal, an isolator, a conductor, a semiconductor, a nanotube, a nanosphere, a nanosheet, a nanofilm, a C₆₀ fullerenes, a fullerene-type concentric graphitic particle, a semiconductor, CdSe, CdS, ZnS, GaAs, InP, nanowires/nanorods such as Si, Ge, SiO_{X}, Ge, O_{X}, nanotubes, single or multiple elements such as carbon, BₓN_{y}, C_{X}, B_{Y}, N_{Z}, MoS₂, and WS₂ and combinations thereof.

12. The method of claim 1, wherein the structural substrate comprises an organic molecule, a peptide, a surfactant, a lipid, a protein, a carbohydrate, a nucleic acid, a metal, a plastic, a monomer, a dimer, a trimer, an oligomer, a polymer, a pharmaceutical, a nutraceutical, a cosmoceutical and combinations thereof.

13. The method of claim 1, wherein the structural substrate is defined as seeding the polymerization reaction and the seeds comprises a emeraldine•HCl nanofiber, a HiPco SWNT, a hexapeptide AcPHF6, a V₂O₅ nanofiber, a polyaniline dimer, polypyrrole dimer and combinations thereof.

14. A product made with the process of claim 1.

15. A molecular polymer comprising a polymer comprising oxidatively coupled monomers, wherein the polymer is formed on or about a molecular seed and the polymer takes on the morphology of the molecular seed.

16. The polymer of claim 15, wherein the polymer is electrically conducting.

17. The polymer of claim 15, wherein the polymer is formed into a film, a sheet, a pill, a powder, a matrix, a fiber, a pad, a filter, a sheet, a sensor, an insert, a cathode, an anode, a passivation, a semiconductor and combinations thereof.

18. The polymer of claim 15, wherein the polymer is a biocompatible, a biodegradable or a bio-degradable and bio-compatible polymer.

19. The polymer of claim 15, wherein the monomers comprises lactic acid, glycolic-lactic acid, glycolic acid, cyanobutylacrylate, propylbutyl, pyrrole, ethylenedioxythiophene and combinations thereof.

20. The polymer of claim 15, further comprising a bioactive compound selected from a drug, a protein, a peptide, a polysaccharide, an oligonucleotide, an antibiotic, an anti-cancer drug, an antigen, an antibody, a bioactive extract, a synthetic organic molecule, and a synthetic inorganic molecule.

21. The polymer of claim 15, further comprising an inorganic molecule, a crystal, a magnet, a metal, an insulator, a conductor, a single crystal semiconductor, a semiconductor, a nanotube, a nanosphere, a nanosheet, a nanofilm, a nanocone, a C₆₀ fullerenes, a fullerene-type concentric graphitic particle, sheet, cone, tube, rod; a semiconductor, CdSe, CdS, ZnS, GaAs, InP, nanowires/nanorods such as Si, Ge, SiO_{X}, Ge, O_{X}, nanotubes, single or multiple elements such as carbon, B_{X}N_{y}, C_{X}, B_{Y}, N_{Z}, MoS₂, and WS₂ and combinations thereof.

22. The polymer of claim 15, further comprising an organic molecule, a peptide, a surfactant, a lipid, a protein, a carbohydrate, a nucleic acid, a metal, a plastic, a monomer, a dimer, a trimer, an oligomer, a polymer, a pharmaceutical, a nutraceutical, a cosmoceutical and combinations thereof.

23. The polymer of claim 15, wherein the molecular seed comprises an oxidant.

24. The polymer of claim 15, wherein the molecular seed comprises an inorganic molecule, a crystal, a magnet, a metal, an insulator, a conductor, a semiconductor, a nanotube, a nanosphere, a nanosheet, a nanofilm, a C₆₀ fullerenes, a fullerene-type concentric graphitic particle, a semiconductor, CdSe, CdS, ZnS, GaAs, InP, nanowires/nanorods such as Si, Ge, SiO_{X}, Ge, O_{X}, nanotubes, single or multiple elements such as carbon, B_{X}N_{y}, C_{X}, B_{Y}, N_{Z}, MoS₂, and WS₂ and combinations thereof.

25. The polymer of claim 15, wherein the molecular seed comprises an organic molecule, a peptide, a surfactant, a lipid, a protein, a carbohydrate, a nucleic acid, a metal, a plastic, a monomer, a dimer, a trimer, an oligomer, a polymer, a pharmaceutical, a nutraceutical, a cosmoceutical and combinations thereof.

26. The polymer of claim 15, wherein the molecular seed is defined as seeding the polymerization reaction and the seeds comprises a emeraldine•HCl nanofiber, a HiPco SWNT, a hexapeptide AcPHF6, a V₂O₅ nanofiber, a polyaniline dimer, polypyrrole dimer and combinations thereof.

27. A polymerization method comprising the step of:
polymerizing in a single step an oxidatively reactive monomer in the presence of an oxidant on or about a molecular seed, wherein the polymer takes on the morphology of the structural substrate.

28. The method of claim 27, wherein the molecular seed comprises an oxidant.

29. The method of claim 27, wherein the oxidatively reactive monomer polymerizes into a bio-compatible polymer.

30. The method of claim 27, wherein the oxidatively reactive monomer polymerizes into a bio-degradable polymer.

31. The method of claim 27, wherein the oxidatively reactive monomer is selected from lactic acid, glycolic-lactic acid, glycolic acid, cyanobutylacrylate, propylbutyl, pyrrole, 3,4-ethylenedioxythiophene,aniline and combinations thereof.

32. The method of claim 27, wherein the polymerization uses one or more of the following polymerizable monomers: ethylene glycol; ethylene oxide; partially or fully hydrolyzed vinyl alcohol; vinylpyrrolidon; ethyloxazoline; ethylene oxide-co-propylene oxide; block copolymers, poloxamers, meroxapols, poloxamines; conductive polymers; methyl, laural, stearyl or butyl methacrylates; vinyl halides; tetrafluroethylene, acrylonitrile, ethylene; methyl, ethyl or butyl acrylates and combinations thereof.

33. The method of claim 27, wherein a second polymer is added to the polymerization reaction selected from natural polymers comprising carboxymethyl cellulose, and hydroxyalkylated celluloses such as hydroxyethyl cellulose and methylhydroxypropyl cellulose, polypeptides, polysaccharides or carbohydrates, polysucrose, hyaluronic acid, dextran, heparan sulfate, chondroitin sulfate, heparin, and alginate, and proteins such as gelatin, collagen, albumin, and ovalbumin, other copolymers, and combinations thereof.

34. The method of claim 27, further comprising the step of adding a bioactive compound.

35. The method of claim 34, wherein the bioactive compound is selected from the group consisting of a drug, a protein, a peptide, a polysaccharide, an oligonucleotide, an antibiotic, an anti-cancer drug, an antigen, an antibody, a bioactive extract, a synthetic organic molecule, and a synthetic inorganic molecule.

36. The method of claim 27, wherein the molecular seed comprises an inorganic molecule, a crystal, a magnet, a metal, an insulator, a conductor, a semiconductor, a nanotube, a nanosphere, a nanosheet, a nanofilm, a C₆₀ fullerenes, a fullerene-type concentric graphitic particle, a semiconductor, CdSe, CdS, ZnS, GaAs, InP, nanowires/nanorods such as Si, Ge, SiO_{X}, Ge, O_{X}, nanotubes, single or multiple elements such as carbon, B_{X}N_{y}, C_{X}, B_{Y}, N_{Z}, MoS₂, and WS₂ and combinations thereof.

37. The method of claim 27, wherein the molecular seed comprises an organic molecule, a peptide, a surfactant, a lipid, a protein, a carbohydrate, a nucleic acid, a metal, a plastic, a monomer, a dimer, a trimer, an oligomer, a polymer, a pharmaceutical, a nutraceutical, a cosmoceutical and combinations thereof.

38. The method of claim 27, wherein the molecular seed is defined as seeding the polymerization reaction and the seeds comprises a emeraldine•HCl nanofiber, a HiPco SWNT, a hexapeptide AcPHF6, a V₂O₅ nanofiber, a polyaniline dimer, polypyrrole dimer and combinations thereof.

39. A product made with the process of claim 27.

40. A method of controlled nanofibril formation comprising polymerizing an oxidatively reactive monomer on or about a template in the presence of an oxidant, wherein the polymer formed takes on the morphology of the structural substrate.

41. A nanofiber comprising a nanofibrillar polymer having a surface area greater than 51 m²/g.

42. The nanofiber of claim 41, wherein the polymer is electrically conducting.

43. The nanofiber of claim 41, wherein the polymer is formed into a film, a sheet, a pill, a powder, a matrix, a fiber, a pad, a filter, a sheet, a sensor, an insert, a cathode, an anode, a passivation, a semiconductor and combinations thereof.

44. The nanofiber of claim 41, wherein the nanofiber is biocompatible.

45. The nanofiber of claim 41, wherein the nanofiber is biodegradable.

46. The nanofiber of claim 41, wherein the polymer comprises a lactic acid, glycolic-lactic acid, glycolic acid, cyanobutylacrylate, propylbutyl, polypyrrole, poly-3,4-ethylenedioxythiophene, and combinations thereof.

47. The nanofiber of claim 41, further comprising a bioactive compound selected from a drug, a protein, a peptide, a polysaccharide, an oligonucleotide, an antibiotic, an anti-cancer drug, an antigen, a bioactive extract, a synthetic organic molecule, and a synthetic inorganic molecule.

48. The nanofiber of claim 41, further comprising an inorganic molecule, a crystal, a magnet, a metal, an insulator, a conductor, a single crystal semiconductor, a semiconductor, a nanotube, a nanosphere, a nanosheet, a nanofilm, a C₆₀ fullerenes, a fullerene-type concentric graphitic particle, a semiconductor, CdSe, CdS, ZnS, GaAs, InP, nanowires/nanorods such as Si, Ge, SiO_{X}, Ge, O_{X}, nanotubes, single or multiple elements such as carbon, B_{X}N_{y}, C_{X}, B_{Y}, N_{Z}, MoS₂, and WS₂ and combinations thereof.

49. The nanofiber of claim 41, further comprising an organic molecule, a peptide, a surfactant, a lipid, a protein, a carbohydrate, a nucleic acid, a metal, a plastic, a monomer, a dimer, a trimer, an oligomer, a polymer, a pharmaceutical, a nutraceutical, a cosmoceutical and combinations thereof.

50. The nanofiber of claim 41, further comprising a targeting moiety.

51. The nanofiber of claim 41, wherein the template is defined further as a sacrificial template.
